(19) United States
(12) Patent Application Publication
(10) Pub. No.: US 2009/0048554 A1
(43) Pub. Date: Feb. 19, 2009

(54) EXTRACORPOREAL CIRCULATION SYSTEM

(76) Inventors: Osvaldo NOGUEIRA SANCHES, Sao Paulo (BR); Nadia MARIA ROCHA MENDES, Sao Paulo (BR)

Correspondence Address: SUGHRUE MION, PLLC, 2100 PENNSYLVANIA AVENUE, N.W., SUITE 800, WASHINGTON, DC, 20037, US

(21) Appl. No.: 11/868,214

(22) Filed: Oct. 5, 2007

(30) Foreign Application Priority Data
Aug. 15, 2007 (BR) 018070053123

(51) Int. Cl. A61M 1/10

(52) U.S. Cl. 604/6.14

(57) ABSTRACT

The present model of utility is related to an extracorporeal circulation system used as an extracorporeal blood circulation system or as a temporary respiratory mechanical support known as ECMO that is quickly changed from one into the other, providing the physician with more time to evaluate the real need to install a long term ECMO.

### DESCRIPTION OF THE INVENTION

The present model of utility is related to an extracorporeal system, that is frequently used either in heart surgeries for extracorporeal circulation of the blood or after the surgery as a temporary respiratory mechanical support.

### DISCLOSURE OF THE INVENTION

It is known by those skilled in the art that the circulation of the blood in the human organism is carried out through the pumping action of the heart. Basically, it is composed of two right chambers and two left chambers, that is, the atria and right and left ventricles. The right atrium receives the venous blood and sends same to the right ventricle, where it is pumped to the lungs for oxygenation. The left atrium receives the blood that returns already oxygenated from the lungs and sends same to the left ventricle, from where it enters in the body circulation system to reach every organ, releasing oxygen to tissues and absorbing carbon dioxide therefrom, finally returning to the right atrium, restarting the circuit.

In heart surgeries, the heart must be freed, thus requiring the use of a system that supports vital cardiac and pulmonary functions, by artificially substituting the heart and the lungs. This is carried out by an extracorporeal circulation system, known as CEC, where the blood of the patient is taken from his body to be purified and oxygenated, and then it is returned to his body.

Such systems comprise several devices that basically consist of a duct loop that connect the patient to a blood reservoir, a pump able to carry the blood flow, an oxygenation chamber provided with a heat exchanger to oxygenate the blood, a device to filter any undesired aggregate and eliminate the air bubbles present in the loop.

The pump replaces the heart by pumping the blood through the loop, while the oxygenation chamber replaces the lungs by oxygenating the blood that returns oxygenated to the patient.

A duct or cava line runs from the patient to the reservoir, and then towards the pump, passing through the oxygenating chamber and then the filter, closing the loop by returning to the patient.

Sometimes, when a surgery is terminated, one cannot be certain if the patient may survive without the help the pump and the oxygenating chamber, either due to a pulmonary deficiency in oxygenating the blood or to the heart itself in pumping the blood. In such situations, the use of a long term extracorporeal respiratory support system (called ECMO) is required. Such a system comprises an oxygenating membrane that helps to oxygenate the blood that is circulating out of the body with the aid of a pump, thus assisting the recovery of the patient in this post-surgery condition.

Disadvantageously, the cost of an ECMO system is high, and despite the uncertainty of the necessity of its use, it is usually decided not to use same in order not to risk the life of the patient, what is always required when it cannot leave the extracorporeal circulation (CEC).

Additionally, the transfer of the extracorporeal circulation system (CEC), used during the surgery, to the post-surgery long term extracorporeal respiratory support system (ECMO) is not immediate. The ECMO requires a minimum time to be assembled, what is not always possible, in view of the emergency of the critical situation the patient is found most of the time.

In order to fulfill such deficiencies found in the art, a multi-functional extracorporeal circulation system was developed that allows an extracorporeal circulation system (CEC) to be changed into a temporary extracorporeal respiratory support system quickly. Such a system is installed quickly, providing the physician with more time to evaluate the real need of installing a long term ECMO.

A temporary ECMO is another object of the present invention that uses the blood of the CEC, thus preventing the need to add blood derivatives, preventing the contamination of the patient's blood.

Another advantage of the temporary ECMO is that it maintains the acid-base and metabolic balance of the patient, thus preventing blood imbalance, besides keeping the normothermia of the patient.

Another object of the present invention is a fast-to-assemble system that is safe as well, because there is a minimum risk of contamination, since it uses the same prime (blood) of the oxygenating chamber. Since it is simple to install, the perfusion professional himself can assemble same without the aid of third parties.

Thus, advantageously, the fast-to-assemble system of the present invention provides the patient with a longer lifetime, thus assuring the surgical team enough time to decide whether to install or not a long term ECMO in the future.

The present system functions as a safety way to the surgeon and the perfusion professional when used in most severe cases where it is suspected that the percentage of surgical success is very small, for example, when the patient's ventricle ejection fraction is lower than 30%, or congenital with VE hypoplasia, T4F of bad anatomy, TGA of bad anatomy, etc.

Schematic figures of a particular embodiment of the invention are given below, whose dimensions and proportions are not necessarily the real ones, since the sole purpose of the figures is to show in a illustrative way the many aspects of the invention, whose protection coverage is determined only by the scope of the attached claims.

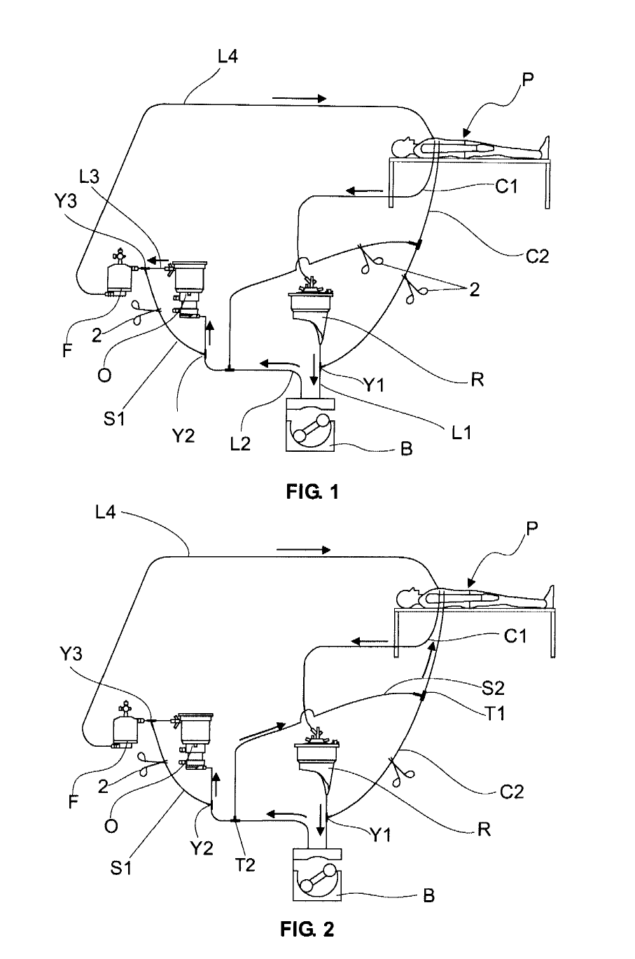

FIG. 1 illustrates a schematic view of the present extracorporeal circulation system applied to a patient (P);

FIG. 2 illustrates a schematic view of the present system with an additional bridge or shunt (S2) to fill the vein (C2);

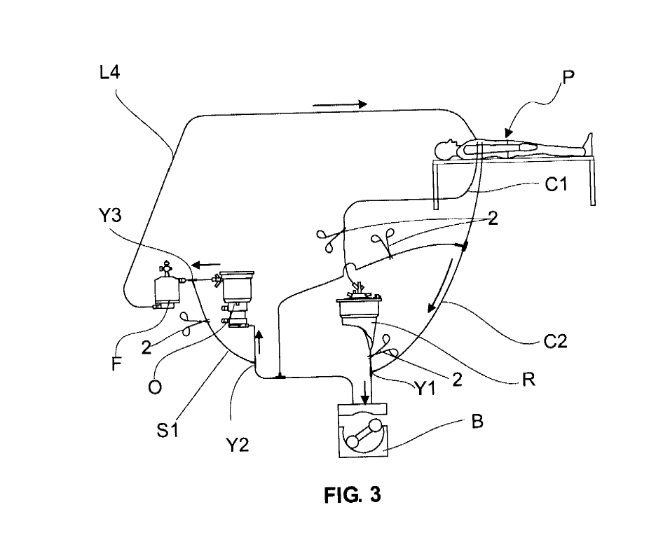

FIG. 3 illustrates a schematic view of the present system applied to a patient (P) in its second function, that is, a temporary extracorporeal respiratory support (ECMO).

As illustrated in the attached figures, it is an extracorporeal circulation system (1) to be used as an extracorporeal circulation system (CEC) or as a temporary extracorporeal respiratory support ECMO).

It is provided with two cava lines (C1, C2); a pump (b) to promote the circulation of the blood; a oxygenating membrane chamber (O) with a heat exchanger coupled thereto to oxygenate the blood, a venous blood reservoir (R), and ducts (L1), (L2), (L3) and (L4) that complete the loop.

Optionally, the system (1) comprises an arterial line filter (F) to eliminate air bubbles and/or impurities.

The pump (B) is any pump suitable for the extracorporeal circulation of blood, such as a peristaltic pump, an arterial roller, a centrifugal pump (e. g., Bio-pump), etc.

The present system (1), when used in extracorporeal circulation (CEC) (illustrated in FIG. 1), has one of its cava lines (C1) in the patient (P), for the transfer of venous blood in the loop to the reservoir (R), the blood running therefrom to the pump (b) through a line or duct (L1), and then towards the oxygenating membrane chamber (O) through the line or duct (L2), and from the oxygenating chamber (O) to the filter (F) through the duct (L3), and finally the oxygenated blood returns to the patient through the arterial duct (L4).

The cava line (C2) is a second cava line for attaining a possible ECMO that runs from the patient for the transfer of venous blood in the system (1). It is connected to the duct (L1) between the reservoir (R) and the pump (B) by means of a “Y” connection (Y1), and is clamped while the CEC is running.

Said system (1) also comprises a bridge known as shunt (S1) that is parallel to the oxygenating membrane chamber, connected to the duct (L2) by means of a “Y” connection (Y2) and to the duct (L3) by means of another “Y” connection (Y3), that is clamped while the CEC is running.

Thus, when the present system is used for a extracorporeal circulation (CEC), it should clamp the third cava line (C2) and the bridge (S1), allowing the blood circulation through the first cava line (C1), reservoir (R), duct (L1), pump (B), duct (L2), oxygenating membrane chamber (O), duct (L3), arterial filter (F) and return to the patient through the arterial duct (L4) (see FIG. 1).

The third cava line (C2) available is clamped while the CEC is running and, preferably, it is only filled with the perfusate when the temporary ECMO is installed. This takes place through a second bridge (S2) from the duct (L2) to the cava line (C2). Said bridge (S2) it is connected to the duct (L2) through a three-way connector (T2) and to the cava line (C2) through another three-way connector (T1), thus allowing the cava line (C2) to be filled with venous blood, in order to reduce the micro-bubbles (see FIG. 2).

Thus, the bridge (S2) is used for filling the cava line (C2), but it should be clamped during the extracorporeal circulation and after the cava line (C2) is filled for accomplishing the temporary ECMO.

In this situation, for the installation of the temporary ECMO, the clamp is removed from the bridge (S2), allowing the blood to run to the cava line (C2), the latter being clamped between the connection (Y1) and the three-way connector (T1), thus preventing the blood from returning from the ducts (L1 and L2) to the oxygenating chamber (O). After the cava line (C2) is filled out, the bridge (S2) is clamped for the temporary ECMO.

As illustrated in FIG. 3, the system (1) used as a temporary extracorporeal respiratory support system (ECMO), where the cava line (C1) and the duct (L1) are clamped between the reservoir (R) and the connection (Y1) and the bridge (S1). Thus, in the loop of the temporary ECMO the blood enters the cava line (C2), passes through the duct (L1), is pumped by the pump (B), runs towards the oxygenating chamber (O) through the duct (L2), runs towards the filter (F) through the duct (L3) and finally returns to the patient (P) through the arterial duct (L4).

The bridge (S1) can also be used for disposing a membrane specific for ECMO, thus turning a temporary ECMO into a long term ECMO (not illustrated), without the need to leave the ECMO.

Therefore, the present system (1) can be used as a loop for the CEC during the heart surgery, and as a temporary ECMO whenever required after the surgery, allowing the perfusion professional to turn the CEC into a temporary ECMO very quickly, with one same loop, thus increasing the chances of the patient to survive.

For such, when performing a CEC to be turned into a ECMO after the cava line (C2) is filled, it suffices remove the clamp (2) to release same for draining the blood to the connector (Y1), the duct (L1) being clamped between said connector (Y1) and the venous blood reservoir (R), and the cava line (C1) being also clamped, thus interrupting the cava line of the CEC.

The present system (1) is simple and makes it possible to the perfusion professional himself trained to perform a CEC to turn same into a temporary ECMO in a matter of minutes, without leaving the CEC, by using the perfusate itself, keeping the metabolic electrolytic balance without altering the adequate blood volume and normothermia of the patient. Thus, any addition of heterologous blood or descompensated hypothermic derivatives is prevented.

A few hours after the temporary ECMO has been installed, the surgical team can decide to install the long term ECMO using a membrane of the ECMO through the bridge (S1), turning a temporary ECMO into a long term ECMO, without interrupting the already installed ECMO of the present invention.

Those skilled in the art will realize, from the description and the drawings shown, several ways to carry out the invention without departing from the scope of the attached claims.

1. An extracorporeal circulation system, characterized by being comprised of two cava lines (C1, C2); two bridges or shunts (S1, S2); a pump (B) to promote the circulation of the blood; a oxygenating membrane chamber (O) with a heat exchanger coupled for oxygenating the blood; a venous blood reservoir (R); and lines or ducts (L1), L2), (L3) an (L4) that complete the loop; wherein one of the cava lines (C1) runs from the patient (P) to the reservoir (R); the duct (L1) runs from the reservoir (T) to the pump (B); the duct (L2) runs from the pump (B) to the oxygenating membrane chamber (O); the duct (L3) of the oxygenating membrane chamber (O) that is connected to (L4) runs to the patient (P); the cava line (C2) comes from the patient and is connected to the duct (L1) through a connection (Y1); the bridge (S1) is parallel to the oxygenating membrane chamber (O), from a connection (Y2) in the duct (L2) to a connection (Y3) in the duct (L3).

2. The extracorporeal circulation system according to claim 1, characterized by comprising an arterial line filter (F) after the oxygenating chamber (O) connected thereto by a duct (L3), the arterial blood duct (L4) in the outlet thereof running to the patient (P).

3. The extracorporeal circulation system according to any of claims 1 or 2, characterized in that the pump (b) is any one of a peristaltic, arterial roller or centrifugal pump.

4. The extracorporeal circulation system according to any of claims 1 to 4, characterized by being a temporary extracorporeal respiratory support system wherein the duct (L1) between the reservoir (R) and the connection (Y1), the cava line (C1) and the bridges (S1) e (S2) are clamped.

5. The extracorporeal circulation system according to any of claims 1 to 3, characterized by being an extracorporeal circulation system wherein the cava line (C2) and the bridges (S1) e (S2) are clamped.
